(19) United States
(12) Patent Application Publication
(10) Pub. No.: US 2026/0248635 A1
(43) Pub. Date: Aug. 27, 2026

(54) URINE COLLECTION DEVICE AND ANTI-BACKFLOW ASSEMBLY FOR A URINE COLLECTION DEVICE

(71) Applicant: Ronald L. Miller, Portland, OR (US)

(72) Inventor: Ronald L. Miller, Portland, OR (US)

(21) Appl. No.: 19/402,481

(22) Filed: Nov. 26, 2025

Related U.S. Application Data
(63) Continuation-in-part of application No. 19/179,727, filed on Apr. 15, 2025.
Application No. 19/179,727 is a continuation of application No. 19/040,783, filed on Jan. 29, 2025, now Pat. No. 12,329,675.

(51) Int. Cl. A61F 5/449; A61F 5/44; A61F 5/443

(52) U.S. Cl. CPC A61F 5/449; A61F 5/4405; A61F 5/443

(57) ABSTRACT

An anti-backflow assembly for a urine collection device comprises a spout housing and an anti-backflow device. The spout housing is removably mountable to extend through an outlet opening of a primary collection element, wherein the spout housing has a spout formed on a distal portion thereof extending away from the collection element. The spout is connectible to a drain tube. The spout housing has a proximal portion for positioning within the collection element in which a recess is defined. The anti-backflow device is removably securable in the recess. The anti-backflow device restricts urine from backflowing from the distal portion of the spout housing towards the proximal portion and into the primary collection element. The anti-backflow device is removable from the recess without removing the spout housing from the primary collection element.

### CROSS-REFERENCE TO RELATED APPLICATIONS

This application is a continuation-in-part of U.S. Patent Application Ser. No. 19/179,727, filed Apr. 15, 2025, which is a continuation of U.S. Patent Application Ser. No. 19/040,783, filed Jan. 29, 2025, now U.S. Pat. No. 12,329,675. The prior applications are incorporated by reference herein in their entirety.

### BACKGROUND

After a bladder removal surgery (cystectomy ileal conduit), a stoma, that allows urine to freely drain from the kidneys, typically projects from the side of the abdomen at the approximate height, or slightly below the height, of the navel.

One commonly used urine collection system to collect urine from a stoma includes a flexible plastic bag or pouch with an opening in a flange that is placed over and around the stoma and is adhered to the skin to form a sealed connection. This bag arrangement is manufactured by many companies, such as Hollister Inc. and Coloplast.

Typically, the flange is integral with the collection pouch and includes an applied adhesive. The adhesive on the flange is protected with a paper or plastic cover. For use, the protective cover is removed to expose the adhesive.

Some conventional collection systems include a separate flange base for connection with a separate collection pouch. Similar to the integral pouch assembly, the separate flange base is attached around and over the stoma with adhesive. The separate collection pouch then attaches to the separate flange base with a removable interlocking mechanical or friction-type seal (similar to a self-sealing plastic bag).

Conventional collection system pouches can have a drainage spout with a normally closed valve that can be opened to drain urine from the collection bag into a toilet, a urinal or a drain.

A length of separate drainage tubing can be attached to the drainage spout valve. The drainage tube can be routed and connected to a separate container (referred to as a “night bag”) that provides greater capacity than the collection pouch alone.

While most of the collection pouches are a single compartment, some urine collection pouch assemblies are divided into two sections with the secondary portion integrated with, but separated from, the primary collection bag. This design typically provides an anti-reflux system between the upper and lower pouches that is claimed to reduce the backflow of urine (at least within the pouch itself). This arrangement may prevent the backflow of urine in some cases but once the lower pouch is full, the anti-reflux system no longer operates and urine then collects in the upper pouch without any protection against backflow. This also increases the chance that the adhesive attaching the pouch to the user weakens and fails because of the additional pressure applied to the upper pouch.

One of the typical issues for a user wearing pants or a garment with a tight waist band or belt is that the collection pouch needs to be worn outside of the belt or waist band. If it is worn inside the belt or waist band, then the pouch's usable volume is effectively restricted to the section above the belt or waist band. With any pressure or deformation of the urine-filled upper part of the collection pouch, the adhesive on the flange holding the pouch to the skin can be compromised, allowing urine to leak from the base of the pouch.

In addition, if unsupported outside of the belt line, the weight of the pouch as it becomes full of urine will affect the performance of the adhesive attachment to the skin, especially while exercising or performing other physical activities.

Without a protective belt-type cover, wearing a garment such as a swimsuit exposes the entire collection pouch.

Along with the packaging and protective paper over the flange adhesive, once the adhesive starts to fail, the entire collection pouch assembly and base is discarded after four to seven days use. This non-compostable paper and plastic is considered medical waste and typically goes to a landfill or in some jurisdictions, requires special disposal, which consumes additional resources.

### SUMMARY

Described below are implementations of a urine collection device and anti-backflow assembly that address some of the problems in the prior art.

According to one implementation, an anti-backflow assembly for a urine collection device having a primary collection element shaped to surround a stoma formed in a person's abdomen and an outlet opening through which collected urine passes, comprises a spout housing and an anti-backflow device. The spout housing is removably mountable to extend through the outlet opening of the primary collection element. The spout housing has a spout formed on a distal portion thereof extending away from the collection element. The spout is connectible to a drain tube. The spout housing has a proximal portion for positioning within the collection element in which a recess is defined. The anti-backflow device is removably securable in the recess of the spout housing. The anti-backflow device restricts urine from backflowing from the distal portion of the spout housing towards the proximal portion and into the primary collection element. The anti-backflow device is removable from the recess of the spout housing without removing the spout housing from the primary collection element.

The anti-backflow assembly can further comprise a retainer engageable with the spout housing to secure the anti-backflow device in the recess.

In some implementations, the spout housing comprises an internal thread in the internal recess adjacent the proximal end, and the retainer has an external thread shaped to engage the internal thread of the internal recess. The retainer is threadedly engageable with the internal thread of the recess with the anti-backflow device received in the recess to secure the anti-backflow device in place within the anti-backflow assembly.

The spout housing can comprise a flange on a proximal side thereof, wherein the flange is configured for contacting an internal surface of the primary collection element when installed in the primary collection element.

The spout housing can comprise a removable retainer positionable to retain the spout housing within the opening in the primary collection element.

The spout housing can comprise an external thread on the distal portion and a spout retainer with an internal thread shaped to engage the external thread. The spout retainer is threadedly engageable with the internal thread to secure the anti-backflow device extending through the outlet opening in the collection element in place.

In some implementations, the anti-backflow device comprises a duckbill-type member formed of a flexible material.

In some implementations, the distal portion of the spout housing comprises a barbed end for connection to the drain tube.

According to another implementation, a urine collection device for a person, comprises a primary collection element shaped to surround a stoma formed in the person's abdomen, the primary collection element having a base, a wall extending from the base and a primary collection volume defined by at least the base and the wall, further comprising a flange extending outwardly from the base and configured for adhesive attachment to the person's skin, and wherein the primary collection element is configured to receive urine from the body opening into the primary collection volume and convey the urine out of the primary collection volume in a first flow direction through an outlet opening in the wall; an anti-backflow assembly removably coupled to the outlet opening of the primary collection element, the anti-backflow assembly having a housing with a proximal portion having a flange for sealing against an inner surface of the primary collection element and an anti-backflow device received in the housing, the anti-backflow assembly having a distal portion with a spout, wherein the anti-backflow assembly is configured to restrict urine from backflowing into the primary collection volume in a second flow direction opposite the first flow direction and compromising the adhesive attachment between the base and the person's skin.

The urine collection device can comprise a drain tube connected to the spout of the anti-backflow assembly. The drain tube can have a working length that extends at least as long as a distance from the body opening in the person's abdomen to a desired secondary collection element location below the person's beltline.

The urine collection device can further comprise a secondary collection element connected to a distal end of the drain tube to collect the urine draining from the drain tube, wherein the secondary collection element is configured for coupling to the person at the desired collection element location.

The spout can comprise a barb fitting with which the drain tube is connected.

The urine collection device can comprise an external thread on the housing and a retainer rotatable to threadedly engage the external thread and couple the anti-backflow assembly to the primary collection element.

The anti-backflow assembly can comprise a recess, and wherein the anti-backflow device is removably secured in the recess. The anti-backflow device can comprise a duckbill-type member formed of a flexible material.

The secondary collection element can be shaped to be suspended from a belt worn by the person, the belt being fitted for wearing adjacent the beltline. The secondary collection element can be configured for being coupled to a leg of the person.

The drain tube can be sized for wearing under a belt or clothing waist band without being compressed by the belt or clothing waist band.

### BRIEF DESCRIPTION OF THE DRAWINGS

FIG. 1 is a front elevation view of a urine collection device having a primary collection element connected to a secondary collection element via a drain tube.

FIG. 2 is a side elevation section view of the primary collection element of FIG. 1.

FIG. 3 is a rear perspective view of the primary collection element of FIG. 1.

FIG. 4 is a side elevation section view showing an alternative adhesive mounting for the primary collection element.

FIG. 5 is an enlarged section view showing the anti-backflow device coupled to the primary collection element.

FIG. 6 is a side section view of an alternative primary collection element having a cover that is removably mounted to a base.

FIG. 7A is a side section view of an alternative primary collection element having an anti-backflow device of a different configuration.

FIG. 7B is a side section view of an alternative arrangement in which the anti-backflow device is removably installed in a housing that is removably connected to a primary collection element or container.

FIG. 7C is an exploded view of the anti-backflow device and housing of FIG. 7B.

FIG. 7D is a side section view of a collection element oriented vertically and showing a separate wall coupled to the flange by one or more retainers on the flange and the anti-backflow device extending through the separate wall.

FIG. 7E is a side section view similar to FIG. 7D showing a separate wall coupled to flange with one or more retainers having separate components.

FIG. 7F is a side section view of a collection device with a separate wall that is coupled to the flange and outlet opening area.

FIG. 8 is a perspective view of the urine collection device showing the secondary collection element configured for wearing on a belt.

FIG. 9 is a perspective view of the urine collection device showing an alternative secondary collection element configured as a tube-shaped container that follows the contour of a belt.

FIG. 10 is a perspective view of a user wearing the urine collection device, with the primary collection device mounted to the surround a stoma in the midsection and the secondary collection device positioned below the beltline, such as in a leg bag configuration that is attached to a leg of the user as shown.

FIG. 11A is a perspective view of the urine collection device in which an alternative secondary collection element is configured to be worn under or incorporated into underwear or another undergarment or garment.

FIG. 11B is a side elevation section view of the urine collection device of FIG. 10 showing the secondary collection element positioned within the underwear.

### DETAILED DESCRIPTION

Described below are implementations of a urine collection device having a primary collection element, an anti-backflow device positioned at or within the primary collection element, a drain tube extending away from the primary collection element and a secondary collection element connected to a distal end of the drain tube.

Referring to FIG. 1, a first implementation of the urine collection device 10 has a primary collection element 40, an anti-backflow device 60, a drain tube 80 and a secondary collection device 90 connected at the distal end of the drain tube 80.

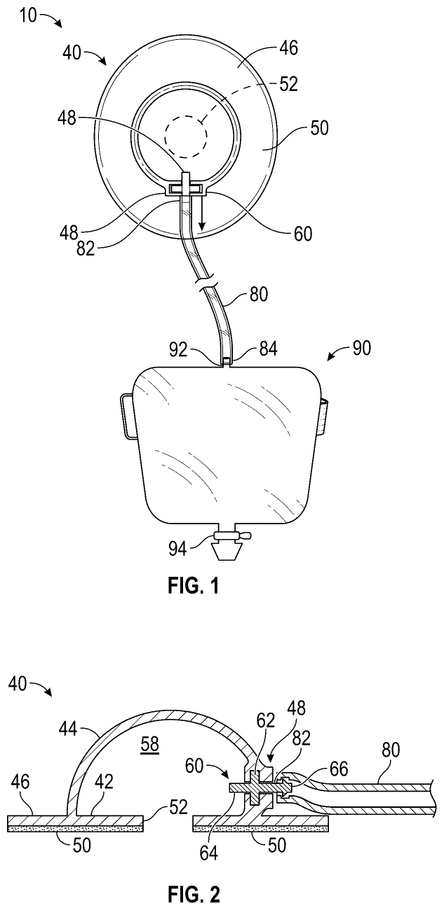

The primary collection element 40 is shaped to surround the stoma or body opening, which is generally located in the patient's midsection. As shown in FIG. 2, the primary collection element 40 has a base 42 and at least one wall 44 that extends away from the base 42. The base 42 and the wall 44 together define a primary collection volume 58. The primary collection volume 58/wall 44 may have a curved outer profile (dome shape) as shown in FIG. 2, a flat outer profile, or another different shape. Although the primary collection element (such as the primary collection element 40) is shown horizontally for convenience of illustration in some of the drawing figures, it is typically worn vertically as illustrated in FIG. 1.

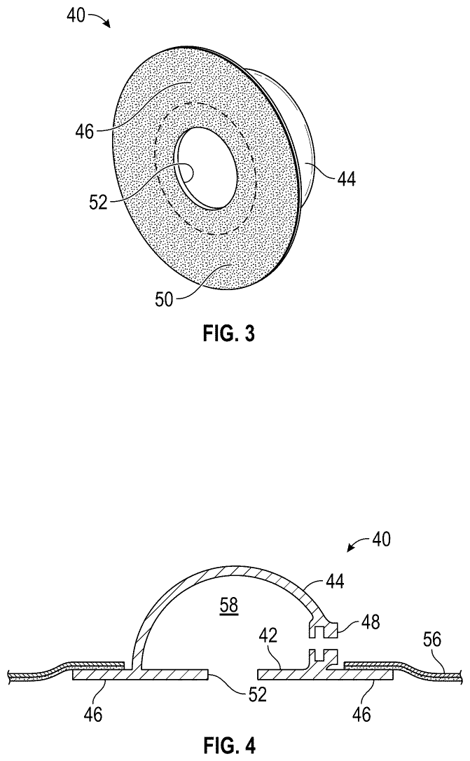

Referring to FIG. 3, which shows a rear side of the primary collection element 40, there is an orifice 52 formed in the base 42 and shaped to encircle the stoma or body opening.

Further, the base may be adjoined by a flange 46 as shown, such as for coupling the primary collection element 40 to the patient. When the urine collection device 10 is worn by the patient, urine is received into the primary collection element 40 via the orifice 52 from the stoma or body opening and initially collects in the primary collection volume 58.

In some embodiments, the primary collection element 40 is coupled to patient using an adhesive 50 between the flange 46 and the patient's skin.

The primary collection element 40 has an outlet opening 48, and the drain tube 80 is connected to the outlet opening 48 (directly or indirectly) to drain urine from the primary collection element 40 to the secondary collection element 90.

The urine collection device 10 preferably has an anti-backflow device 60 that is positioned at, or within, or very close to, the primary collection element 40. The anti-backflow device 60 functions to permit relatively unrestricted flow of urine out of the primary collection element 40 and through the drain tube 80 distally (i.e., away from the primary collection element 40), but blocks or at least substantially restricts flow in an opposite proximal direction (i.e., towards the primary collection element 40). The anti-backflow device is also known as a one-way valve or a check valve. In this way, the anti-backflow device 60 prevents urine in the tube 80 and/or the secondary collection element 90 from backing up into the primary collection element 40, which would increase pressure in the primary collection element 40 and could weaken the adhesive bond between the primary collection element 40 and user's skin (resulting in leaking of urine) and/or impeding the flow of urine from the stoma. The tendency of urine in the drain tube 80 and/or the secondary collection element 90 to backflow toward the primary collection element 40 can be caused by movement, e.g., such as to a sitting or laying position, or another action that exerts pressure on and/or deforms the drain tube and/or the secondary collection element, such as contact with an object.

Referring to FIG. 2, the anti-backflow device has a body 62 with a first end 64 and a second end 66, with the second end being positioned downstream of the first end 64 in the flow direction.

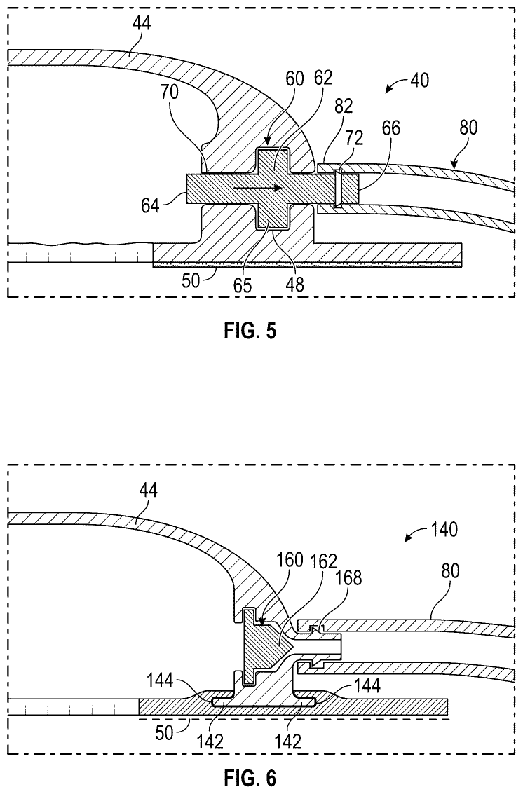

The outlet opening 48 in the wall 44 of the primary collection element 40 can be shaped to receive at least a portion of the body 62 of the anti-backflow device 60. FIG. 4 shows the primary collection element 40 without the anti-backflow device 60 installed to illustrate the shape of the outlet opening 48. Additionally, it is also possible to arrange some or all of the anti-backflow device 60 to protrude from the primary collection element 40 (FIGS. 2 and 5). Alternatively, the anti-backflow device 60 can be positioned interiorly of the wall 44, i.e., within the primary collection volume 58.

Referring to FIG. 4, the primary collection element 40 is shown in greater detail. The body 62 of the anti-backflow device 60 is formed with a midsection 65 having a greater dimension than the first end 64 or the second end 66. The body 62 is shown in its installed position within the outlet opening 48 with the second end 66 protruding outwardly beyond the wall 44. The anti-backflow device 60 can be formed in the installed position as shown with the primary collection element 40, or it can be a separate component that is inserted into the primary collection element 40. For example, if the primary collection element 40 is formed of silicone or a similar resilient material, then it can be stretched as the anti-backflow device 60 is inserted into the installed position.

In the illustrated implementation, the anti-backflow device 60 forms part of the connection to the drain tube 80. Specifically, a first end 82 of the drain tube 80 is fitted to an exterior of the second end 66 of the anti-backflow device 60. The second end 66 may be sized and/or shaped to provide an appropriate friction fit that tends to prevent leakage and also retains the drain tube 80 in a connected state over the useful life of the device. For example, the second end 66 may define a Luer fitting (e.g., a slight taper) for establishing an appropriate connection with the drain tube 80.

Alternatively, as shown in FIG. 5, the second end 66 can include a barb fitting 72 to improve the connection to the to the drain tube 80. The first end 64 can also include a barb fitting 72. Instead of or in addition to the barb fitting 72, the second end 66 could be formed with one or more ribs to create a rib fitting.

As another alternative, an anti-backflow device 160 in the form of a duckbill device as shown in FIG. 6 can be used. As also shown in FIG. 6, a primary collection element 140 is formed with a projecting extension or spout 168, and the drain tube 80 is connected to the spout 168. The spout 168 may have a barb connection as shown, or a friction-fit, Luer, rib or other type of connection can be used.

Suitable anti-backflow devices include the Nordson check valve SCV 06265, with a barb, Luer or other fitting, available from Nordson Medical. Also, suitable medical grade duckbill-type check valves are available from Vernay (www. vernay. com).

In the primary collection element 140 of FIG. 6, the dome-shaped portion (defined by the wall 40) is removably secured to the base, such as by an interlocking seal formed by a mating insert 142 inserted into a mating groove 144 or another suitable resealable connection. Among other benefits, the resealable connection allows for disposing of the base that has the adhesive when it is damaged or no longer usable and reusing the dome-shaped portion.

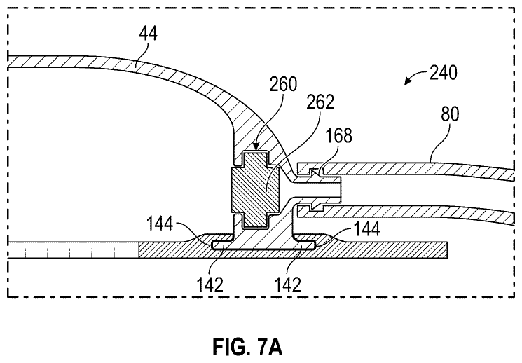

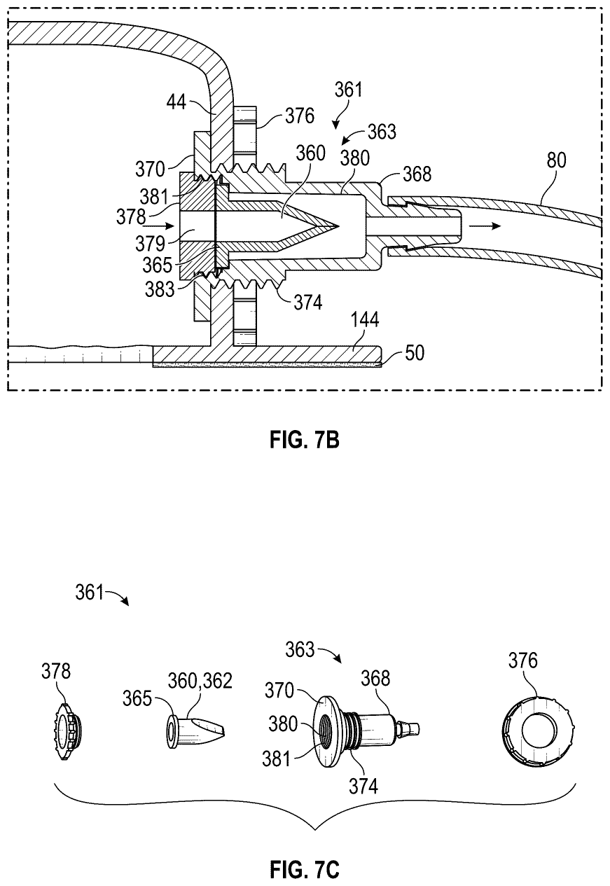

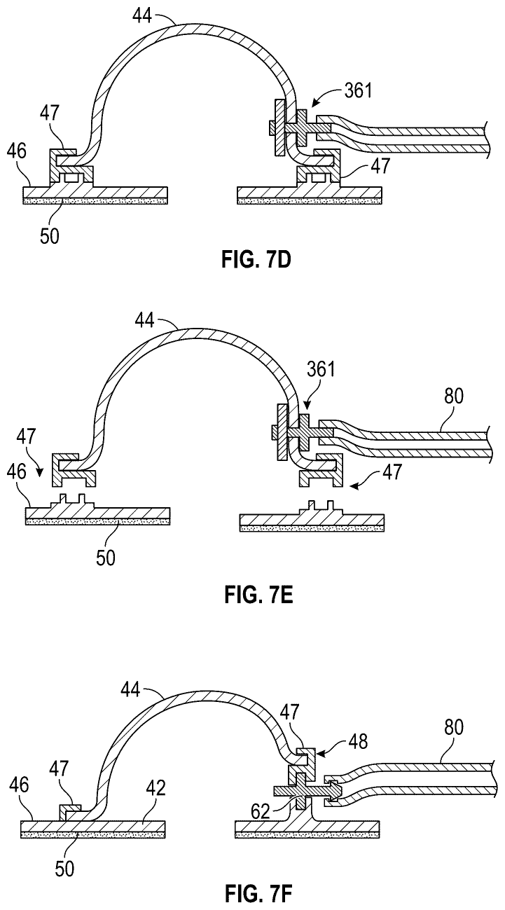

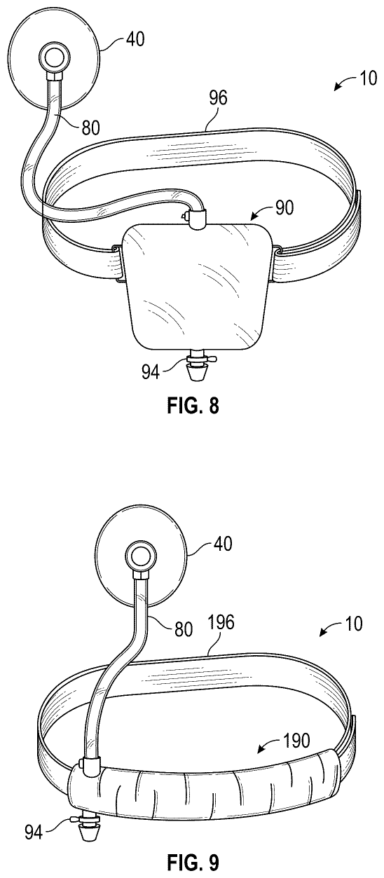

In another implementation, a primary collection element 240 as shown in FIG. 7A is similar to the primary collection element 140, but it has an anti-backflow device 260 that is formed as a check valve or another similar type of valve that can be cast into the primary collection element 240 during manufacture or otherwise formed with it, or inserted into a space reserved for the anti-backflow device 260.

In still other implementations, the anti-backflow device or a portion thereof may be formed as one piece with one or more of the walls of the device, such as the wall 40. The anti-backflow device 260 may be formed of the same material(s) as the primary collection element 240, or of different material(s).

According to other implementations, the anti-backflow device is removably installed in a housing, and the housing is removably installed in a primary collection element or any other type of container. As described above, the primary collection element can include adhesive 50 (such as two-sided adhesive tape or other adhesive, and optional removable protection layer (e.g., paper)) to allow the primary collection element to be attached to the patient. Referring to FIGS. 7B and 7C, an anti-backflow assembly 361 has a spout housing 363 with a spout 368 formed at a distal end thereof and a recess 380 defined within the spout housing 363 and accessible from a proximal end opposite the distal end. On a distal portion thereof, the spout 368 can have a barb fitting as shown, or any other suitable end configuration (friction-fit, Luer, rib or other type of connection).

The recess 380 in the spout housing 363 is sized to receive an anti-backflow device 360, such as a duckbill device. The anti-backflow device 360 has a body 362 and is retained in the recess 380 during normal use, but is removable, such as for cleaning or replacement. The anti-backflow device 360 can be interfitted or removably engageable with the recess 380, such as with complementary projections and depressions, or a threaded arrangement. In the illustrated implementation, the recess 380 has an internal thread 381. A retainer 378 is configured with an external thread 383 to threadedly engage with the internal thread 381 to retain the anti-backflow device 360 in place within the recess 380.

The retainer 378 has a central opening or bore 379 that is sized to allow urine to flow from the primary collection element to the anti-backflow device 360 as indicated by the arrows with minimal added resistance. The retainer 378 can be threaded into the recess 380 by hand. In some implementations, the retainer 378 can be provided with a texture, depressions or protrusions to provide greater friction during installation and/or removal, as well as to facilitate light tool use in some situations.

As stated, the spout housing 363 is removably installed (or mounted) to extend through the wall 44 in the primary collection element 40 as shown. In the illustrated implementation, the spout housing 363 can have an outwardly extending flange 370, which can be positioned at the second end as shown, or inwardly of the second end. The spout housing 363 has a threaded section 374. A spout housing retainer 376 has an internal thread configured to engage with the threaded section 374. In use, the spout housing 363 is inserted through an opening in the wall 44 with the flange 370 bearing against one side of the wall 44 (i.e., the proximal side) and the spout retainer 376 is threaded onto the threaded section 374 until it is tightened against the other side of the wall 44 (i.e., the distal side), thereby forming a removable connection that is leak free.

With the anti-backflow assembly 361, the anti-backflow device 360 is removable by removing the retainer 378 to access the recess 380, without the need to remove the spout housing 363 or change its connection to the wall 44. In this way, the anti-backflow device 360 can be removed for cleaning, or replaced with a new device of the same type or a different type. Waste is reduced because the anti-backflow device can be changed independent of the other components, which can be reused. In some cases, it may be necessary to access the retainer from within the primary collection element (or other container to which the anti-backflow assembly is attached).

Similarly, the anti-backflow assembly 361 provides for more flexible mounting options to a primary collection element 40, as well as any other type of container. Specifically, only a suitably sized opening in the wall 44 or a wall of the container is required. The anti-backflow assembly 361 is easily mounted by guiding its distal portion through the opening and threading the spout retainer 376 against the wall (or surface) surrounding the opening such that the flange 370 is in contact with the opposite side of the wall (or another proximal surface). If desired, the anti-backflow assembly 361 can be easily removed, such as for re-use with a new or different container, by unthreading the spout retainer 376 and withdrawing it proximally from the opening. Providing for re-use of the anti-backflow assembly 361 in this way also helps reduce waste.

In the illustrated implementation, the body 362 of the anti-backflow device 360 can include an enlarged shoulder 365 as shown. One suitable class of anti-backflow devices are one-way duckbill valves, such as the Vernay V246110200 duckbill valve, which is described as a single-component, in-line, one-way check valve that is self-sealing. Such duckbill valves are ideal for applications requiring immediate forward flow with a straight-through flow path. Such a duckbill valve is typically made of silicone or another similar material.

In the examples above and below, the urine collection device, and more specifically any collection element thereof, can have any suitable wall or material thickness. In some implementations, the collection element wall or material may be 0.15 to 0.30 mm in thickness, depending upon the intended application. For example, to emulate conventional disposable collection elements (bags), the material or wall thickness may be in the thinner part of this range. Similarly, a collection element for an application that may experience greater wear or stresses, e.g., an ankle bag, may have a wall or material thickness in the thicker part of this range. Also, the wall or material thickness need not be constant and may vary at different locations on the collection element. The wall or material thickness may be thin enough that the collection element collapses under its own weight when empty, or it may thick enough to be self-supporting when empty.

In implementations of the anti-backflow assembly 361, the wall or material thickness is sufficient in at least the area surrounding the opening to allow the assembly to seal against the surface of the material around the opening without leaking or tearing (i.e., from the action of tightening the spout retainer 376 against the surface of the material).

FIGS. 7D-7F show examples of urine collection devices in which the collection element is illustrated as being made of thinner material. Optionally, the wall 44 may be a separate component.

In FIG. 7E, the wall 44 of the collection element device is shown in having an irregular shape in cross section when empty (i.e., with sags and/or folds) because the wall 44 is thin and may not be self-supporting. The collection element as depicted is in a vertical orientation, with upper and lower ends of the wall 44 coupled to the flange 46, such as with a retainer 47. The flange 46 of FIGS. 7D-7F typically has a greater thickness than the wall 44. The flange 46 has the adhesive 50 for coupling the collection device to the patient. In FIG. 7D, the anti-backflow assembly 361 is mounted through the wall 44.

In FIG. 7E, the retainer (or retainers) 47 are shown as being formed as one piece with the flange 46. As shown in FIG. 7E, the retainer (or retainers) 47 may have multiple components, including a component that is coupled to the edge of the wall 44 and a component attached to the flange 46.

In FIG. 7F, the flange 46 and the outlet opening 48 are formed as one piece, and the wall 44 is coupled to the flange 46 and the area around the outlet opening 48 with the retainer or retainers 47. Similar to the implementation of FIG. 2, the outlet opening 48 is formed to receive and retain the anti-backflow device 60.

FIG. 8 is an illustration of the urine collection device 10 with the secondary collection element 90 configured for wearing on a belt 96. The belt 96 is intended for positioning around the waist (or belt line) of the user, and at a height below the stoma (when the user is upright). In this way, the flow of urine from the primary collection element 40 through the drain tube 80 and to the secondary collection element 90 is generally downward and thus assisted by gravity.

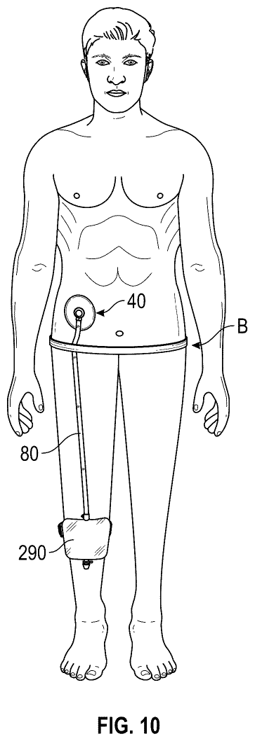

FIG. 9 is an illustration of a secondary collection element 190 that is also belt-worn, but is shaped to have a tube-like configuration that follows the contour of the belt and the user's waist to provide greater comfort and ease of positioning, particularly in seated positions. FIG. 10 is an illustration of a secondary collection element 290 that is configured to be mounted to a leg of the user (which is also below the belt line when the user is upright). Although the secondary collection element 290 is shown mounted to the lower leg, it could also be mounted to the upper leg.

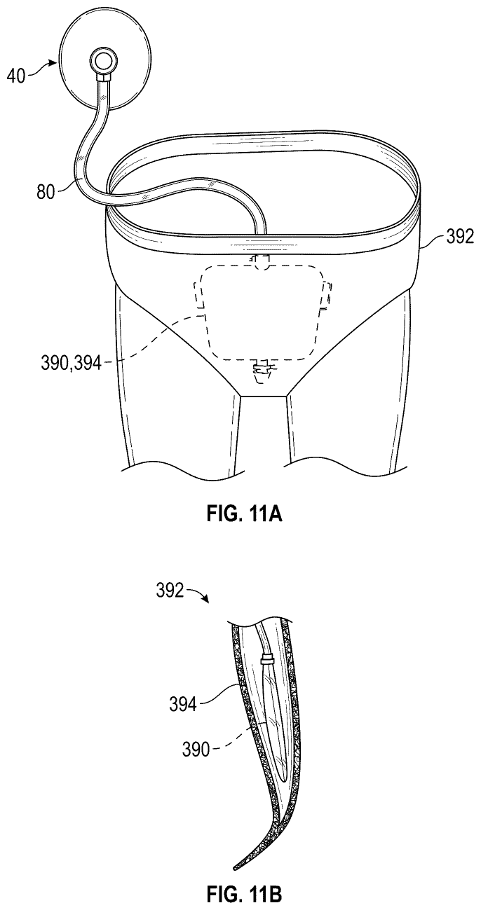

FIGS. 11A and 11B show a secondary collection element 390 configured for wearing in conjunction with a garment, such as underwear 392 or another type of garment. The secondary collection element 390 may be simply inserted into the underwear 392, or the underwear may be fitted with an internal or external pocket. In the illustrated implementation, an internal pocket 394 within the underwear 392 is shaped to receive the secondary collection element 390.

In the above implementations, the volumetric capacity of the primary collection element can be smaller than the volumetric capacities of conventional bags or pouches. A smaller primary collection element is lower in weight and exerts less pressure on the adhesive and skin.

Also, any of the described anti-backflow devices can be configured to be replaceable, such as by removing a used or damaged anti-backflow device and inserted a replacement, particularly in implementations where the primary collection element is formed of a relatively flexible material.

In some implementations, the primary collection element can be cleaned for reuse.

In view of the many possible embodiments to which the principles of the disclosed invention may be applied, it should be recognized that the illustrated embodiments are only preferred examples and should not be taken as limiting the scope of protection. Rather, the scope of protection is defined by the following claims. I therefore claim as my invention all that comes within the scope and spirit of these claims.

1. An anti-backflow assembly for a urine collection device having a primary collection element shaped to surround a stoma formed in a person's abdomen and an outlet opening through which collected urine passes, the anti-backflow assembly comprising:
a spout housing removably mountable to extend through the outlet opening of the primary collection element, wherein the spout housing has a spout formed on a distal portion thereof extending away from the collection element, wherein the spout is connectible to a drain tube, and wherein the spout housing has a proximal portion for positioning within the collection element in which a recess is defined;
an anti-backflow device removably securable in the recess of the spout housing, the anti-backflow device restricting urine from backflowing from the distal portion of the spout housing towards the proximal portion and into the primary collection element; and
wherein the anti-backflow device is removable from the recess of the spout housing without removing the spout housing from the primary collection element.

2. The anti-backflow assembly of claim 1, further comprising a retainer engageable with the spout housing to secure the anti-backflow device in the recess.

3. The anti-backflow assembly of claim 1, wherein the spout housing comprises an internal thread in the internal recess adjacent the proximal end, further comprising a retainer having an external thread shaped to engage the internal thread of the internal recess, wherein the retainer is threadedly engageable with the internal thread of the recess with the anti-backflow device received in the recess to secure the anti-backflow device in place within the anti-backflow assembly.

4. The anti-backflow assembly of claim 1, wherein the spout housing comprises a flange on a proximal side thereof, wherein the flange is configured for contacting an internal surface of the primary collection element when installed in the primary collection element.

5. The anti-backflow assembly of claim 1, wherein the spout housing comprises a removable retainer positionable to retain the spout housing within the opening in the primary collection element.

6. The anti-backflow assembly of claim 1, wherein the spout housing comprises an external thread on the distal portion and a spout retainer with an internal thread shaped to engage the external thread, wherein the spout retainer is threadedly engageable with the internal thread to secure the anti-backflow assembly extending through the outlet opening in the collection element in place.

7. The anti-backflow assembly of claim 1, wherein the anti-backflow device comprises a duckbill-type member formed of a flexible material.

8. The anti-backflow assembly of claim 1, wherein the distal portion of the spout housing comprises a barbed end for connection to the drain tube.

9. The anti-backflow assembly of claim 1, wherein the anti-backflow device prevents urine from backflowing into the primary collection volume and increasing pressure within the primary collection element.

10. A urine collection device for a person, comprising:
a primary collection element shaped to surround a stoma formed in the person's abdomen, the primary collection element having a base, a wall extending from the base and a primary collection volume defined by at least the base and the wall, further comprising a flange extending outwardly from the base and configured for adhesive attachment to the person's skin, and wherein the primary collection element is configured to receive urine from the body opening into the primary collection volume and convey the urine out of the primary collection volume in a first flow direction through an outlet opening in the wall; and
an anti-backflow assembly removably coupled to the outlet opening of the primary collection element, the anti-backflow assembly having a housing with a proximal portion having a flange for sealing against an inner surface of the primary collection element and an anti-backflow device received in the housing, the anti-backflow assembly having a distal portion with a spout, wherein the anti-backflow assembly is configured to restrict urine from backflowing into the primary collection volume in a second flow direction opposite the first flow direction and compromising the adhesive attachment between the base and the person's skin.

11. The urine collection device of claim 10, further comprising a drain tube connected to the spout of the anti-backflow assembly.

12. The urine collection device of claim 11, wherein the drain tube has a working length that extends at least as long as a distance from the body opening in the person's abdomen to a desired secondary collection element location below the person's beltline.

13. The urine collection device of claim 11, further comprising a secondary collection element connected to a distal end of the drain tube to collect the urine draining from the drain tube, wherein the secondary collection element is configured for coupling to the person at the desired collection element location.

14. The urine collection device of claim 11, wherein the spout comprises a barb fitting with which the drain tube is connected.

15. The urine collection device of claim 11, further comprising an external thread on the housing and a retainer rotatable to threadedly engage the external thread and couple the anti-backflow assembly to the primary collection element.

16. The urine collection device of claim 10, wherein the anti-backflow device comprises a recess, and wherein the anti-backflow device is removably secured in the recess.

17. The urine collection device of claim 10, wherein the anti-backflow device comprises a duckbill-type member formed of a flexible material.

18. The urine collection device of claim 13, wherein the secondary collection element is shaped to be suspended from a belt worn by the person, the belt being fitted for wearing adjacent the beltline.

19. The urine collection device of claim 13, wherein the secondary collection element is configured for being coupled to a leg of the person.

20. The urine collection device of claim 11, wherein the drain tube is sized for wearing under a belt or clothing waist band without being compressed by the belt or clothing waist band.
